(19) United States
(12) Patent Application Publication
(10) Pub. No.: US 2010/0305442 A1
(43) Pub. Date: Dec. 2, 2010

(54) SYSTEMS AND METHODS FOR IMPLEMENTING A DATA MANAGEMENT SYSTEM FOR CATHETER-BASED IMAGING SYSTEMS

(75) Inventors: Michael J. Tierney, Pleasanton, CA (US); Lauretta A. Iwamasa, Newark, CA (US); Joel R. Brey, Shoreview, MN (US)

Correspondence Address: Boston Scientific Corporation;c/o Frommer Lawrence & Haug LLP, 745 Fifth Avenue, New York, NY, 10151, US

(73) Assignee: Boston Scientific SciMed, Inc., Maple Grove, MN (US)

(21) Appl. No.: 12/475,193

(22) Filed: May 29, 2009

(51) Int. Cl. A61B 8/00; G06F 12/00; G06F 11/07

(52) U.S. Cl. 600/443; 600/466; 711/100; 714/25; 711/E12.001; 714/E11.029

(57) ABSTRACT

A method of managing catheter data for a catheter-based imaging system includes coupling a catheter to a control module. The catheter includes a memory structure that includes catheter management data. The control module includes a processor. The catheter management data is accessed from the memory structure using the processor. Patient tissue is imaged using control module settings that are selected based, at least in part, on the accessed catheter management data. At least one image is displayed based, at least in part, on the imaged patient tissue.

### TECHNICAL FIELD

The present invention is directed to the area of catheter-based imaging systems and methods of making and using the systems. The present invention is also directed to catheter-based imaging systems having memory structures disposed on catheters that contain catheter management data that is accessible by data processors coupled to corresponding control modules.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

Intracardiac echocardiography (“ICE”) is another ultrasound imaging technique with proven capabilities for use in diagnosing intravascular diseases and disorders. ICE uses acoustic signals to image patient tissue. Acoustic signals emitted from an ICE imager disposed in a catheter are reflected from patient tissue and collected and processed by a coupled ICE control module to form an image.

### BRIEF SUMMARY

In one embodiment, a method of managing catheter data for a catheter-based imaging system includes coupling a catheter to a control module. The catheter includes a memory structure that includes catheter management data. The control module includes a processor. The catheter management data is accessed from the memory structure using the processor. Patient tissue is imaged using control module settings that are selected based, at least in part, on the accessed catheter management data. At least one image is displayed based, at least in part, on the imaged patient tissue.

In another embodiment, a computer-readable medium has processor-executable instructions for reading data from a memory structure disposed on a catheter. The processor-executable instructions, when installed onto a device enable the device to perform actions, include accessing catheter management data from the memory structure. The processor-executable instructions further include imaging patient tissue using control module settings that are selected based, at least in part, on the accessed catheter management data. The processor-executable instructions also include displaying at least one image based, at least in part, on the imaged patient tissue.

In yet another embodiment, a catheter-based imager includes at least one imager and a memory structure disposed in a catheter that is at least partially insertable into a patient. The at least one imager and the memory structure are each coupled to a control module. The catheter-based imager includes a processor in communication with the control module. The processor is for executing processor-readable instructions that enable actions, including accessing catheter management data from the memory structure. The processor-readable instructions further enable imaging patient tissue using control module settings that are selected based, at least in part, on the accessed catheter management data. The processor-readable instructions also enable displaying at least one image based, at least in part, on the imaged patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of a catheter and a corresponding control module of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic view of one embodiment of an intravascular ultrasound system that includes a data management system, according to the invention;

FIG. 5 is a schematic perspective view of one embodiment of a memory structure disposed on a hub of a catheter, according to the invention;

FIG. 6 is a flow diagram of one exemplary embodiment of a data management communication procedure between a memory structure and a data processor of a catheter-based imaging system, according to the invention; and

FIGS. 7A-7B collectively illustrate a flow diagram of one exemplary embodiment of a data management communication procedure between a memory structure and a data processor of a catheter-based imaging system, according to the invention.

### DETAILED DESCRIPTION

The present invention is directed to the area of catheter-based imaging systems and methods of making and using the systems. The present invention is also directed to catheter-based imaging systems having memory structures disposed on catheters that contain catheter management data that is accessible by data processors coupled to corresponding control modules.

The methods, systems, and devices described herein may be embodied in many different forms and should not be construed as limited to the embodiments set forth herein. Accordingly, the methods, systems, and devices described herein may take the form of an entirely hardware embodiment, an entirely software embodiment or an embodiment combining software and hardware aspects. The methods described herein can be performed using any type of computing device, such as a computer, that includes a processor or any combination of computing devices where each device performs at least part of the process.

Suitable computing devices typically include mass memory and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

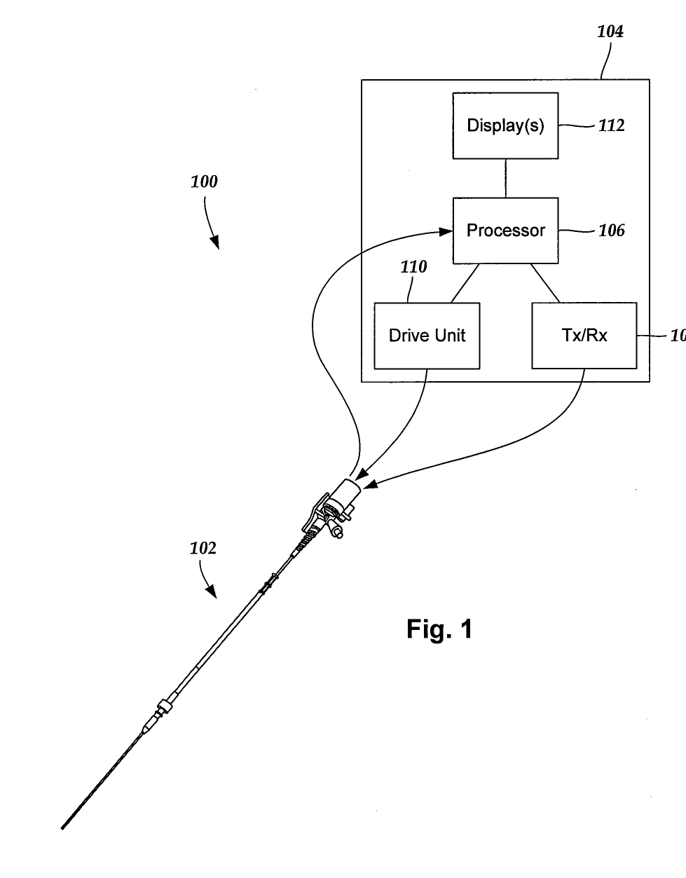

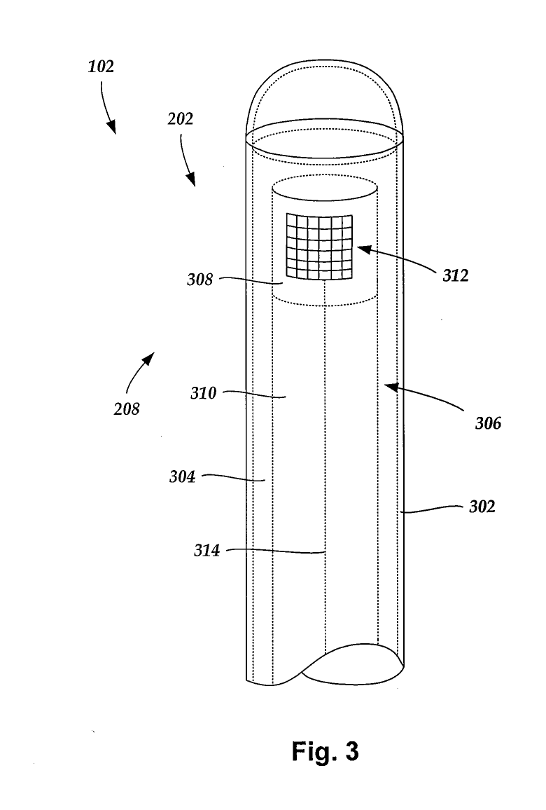

FIG. 1 illustrates schematically one embodiment of an IVUS imaging system 100. The IVUS imaging system 100 includes a catheter 102 that is coupleable to a control module 104. The control module 104 may include, for example, a processor 106, an ultrasound transmitter and receiver 108, a motor 110, and one or more displays 112. In at least some embodiments, the ultrasound transmitter 108 forms electric pulses that may be input to one or more transducers (312 in FIG. 3) disposed in the catheter 102. In at least some embodiments, mechanical energy from the motor 110 may be used to drive an imaging core (306 in FIG. 3) disposed in the catheter 102. In at least some embodiments, tissue reflections from electric pulses transmitted from the one or more transducers (312 in FIG. 3) may be received and forwarded as input to the processor 106 for processing. In at least some embodiments, the processed reflections from electric pulses delivered to one or more transducers (312 in FIG. 3) may be displayed as one or more images on the one or more displays 112.

In at least some embodiments, the processor 106 may also be used to control the functioning of one or more of the other components of the control module 104. For example, the processor 106 may be used to control at least one of the frequency, amplitude, repetition rate, or duration of the electrical pulses transmitted from the ultrasound transmitter 108, the receive gain, sampling rate, filter characteristics, or the signal processing of the received signal 108, the rotation rate of the imaging core (306 in FIG. 3) by the motor 110, the velocity or length of the pullback of the imaging core (306 in FIG. 3) by the motor 110, or one or more properties of one or more images formed on the one or more displays 112.

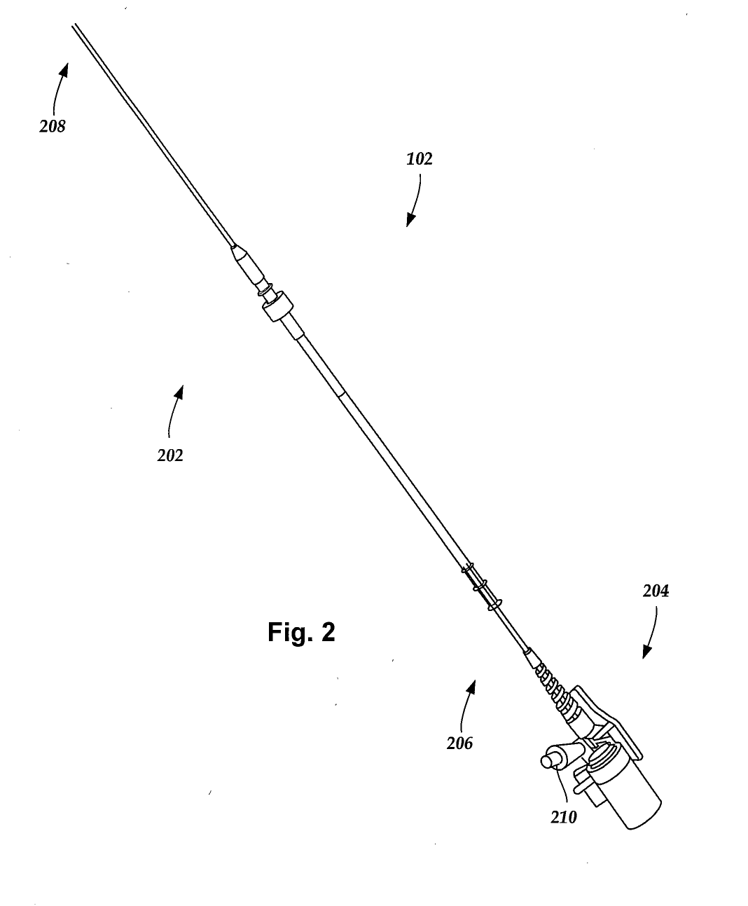

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the catheter 102 does not use a flush port 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. In at least some embodiments, the one or more transducers 312 are configured into an annular arrangement. In at least some embodiments, the one or more transducers 312 are fixed in place and do not rotate.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like)

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received. In at least some embodiments, the rotation of the imaging core 306 is driven by the motor 110 disposed in the control module (104 in FIG. 1).

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112. In at least some embodiments, the one or more transducers 312 are fixed in place and do not rotate during an imaging procedure. In at least some embodiments, at least one of the imaging core 306 or the one or more transducers 312 are manually rotated.

In at least some embodiments, the imaging core 306 may also move longitudinally along the blood vessel within which the catheter 102 is inserted so that a plurality of cross-sectional images may be formed along a longitudinal length of the blood vessel. In at least some embodiments, during an imaging procedure the one or more transducers 312 may be manually retracted (i.e., pulled back) along the longitudinal length of the catheter 102. In at least some embodiments, the catheter 102 includes at least one telescoping section that can be manually retracted during pullback of the one or more transducers 312. In at least some embodiments, the motor 110 drives a pullback mechanism that retracts the imaging core 306 within the catheter 102. In at least some embodiments, the motorized pullback distance of the imaging core is at least 5 cm. In at least some embodiments, the motorized pullback distance of the imaging core is at least 10 cm. In at least some embodiments, the motorized pullback distance of the imaging core is at least 15 cm. In at least some embodiments, the motorized pullback distance of the imaging core is at least 20 cm. In at least some embodiments, the motorized pullback distance of the imaging core is at least 25 cm.

In at least some embodiments, when the imaging core 306 is retracted while rotating, the images collectively form a continuous spiral shape along a blood vessel. In at least some embodiments, when the imagine core 306 is retracted while rotating, a stepper motor, or brushed DC motor, or brushless DC motor may be used to pull back the imaging core 306. The use of any of these motors can pull back the imaging core 306 a short distance and stop long enough for the one or more transducers 306 to capture an image before pulling back the imaging core 306 another short distance and again capturing another image, and so on, either with or without being rotated.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 100 MHz.

In at least some embodiments, one or more conductors 314 electrically couple the transducers 312 to the control module 104 (see e.g., FIG. 1). In at least some embodiments, the one or more conductors 314 extend along a longitudinal length of the rotatable driveshaft 310.

In at least some embodiments, the catheter 102 with one or more transducers 312 mounted to the distal end 208 of the imaging core 308 may be inserted percutaneously into a patient via an accessible blood vessel, such as the femoral artery, at a site remote from the selected portion of the selected region, such as a blood vessel, to be imaged. The catheter 102 may then be advanced through the blood vessels of the patient to the selected imaging site, such as a portion of a selected blood vessel.

Catheter-based imaging systems (e.g., IVUS, ICE, or the like) are commonly used to diagnose patient diseases and disorders. Unfortunately, not all catheters are compatible with all control modules. Some types of catheters may provide better, more, fewer, or limited imaging capabilities compared to other types of catheters for a particular imaging procedure or for a particular type of control module. Additionally, although some types of catheter-based imaging systems may have adjustable settings to improve imaging, little to no information about a given catheter, such as the operational attributes and parameters of the given catheter, might be known to a user of the catheter. In some cases, the coupled catheter may have exceeded its design lifetime and may be unsafe or more prone to unexpected failure.

A data management system can be implemented for a catheter-based imaging system. In at least some embodiments, the data management system can improve safety operability, or other characteristics by providing catheter management data for a catheter that is accessibly to a control module when the catheter is coupled to the control module. In at least some embodiments, the accessed catheter management data can be used to improve image quality by providing operational attributes or parameters for the catheter that can be used to adjust one or more imaging settings of the control module. In at least some embodiments, the accessed catheter management data can be used to facilitate troubleshooting catheter failure by recording operational conditions (and causes of failure) during an imaging procedure.

The data management system includes a memory structure disposed on the catheter and a data processor configured and arranged to access catheter management data embedded in the memory structure. The memory structure may be disposed anywhere on the catheter (102 in FIG. 1). In at least some embodiments, the memory structure is disposed on the hub 204 of the catheter 102. In some embodiments, the data processor is disposed on the control module 104. In other embodiments, the data processor is disposed on a peripheral device coupled to the control module (104 in FIG. 1).

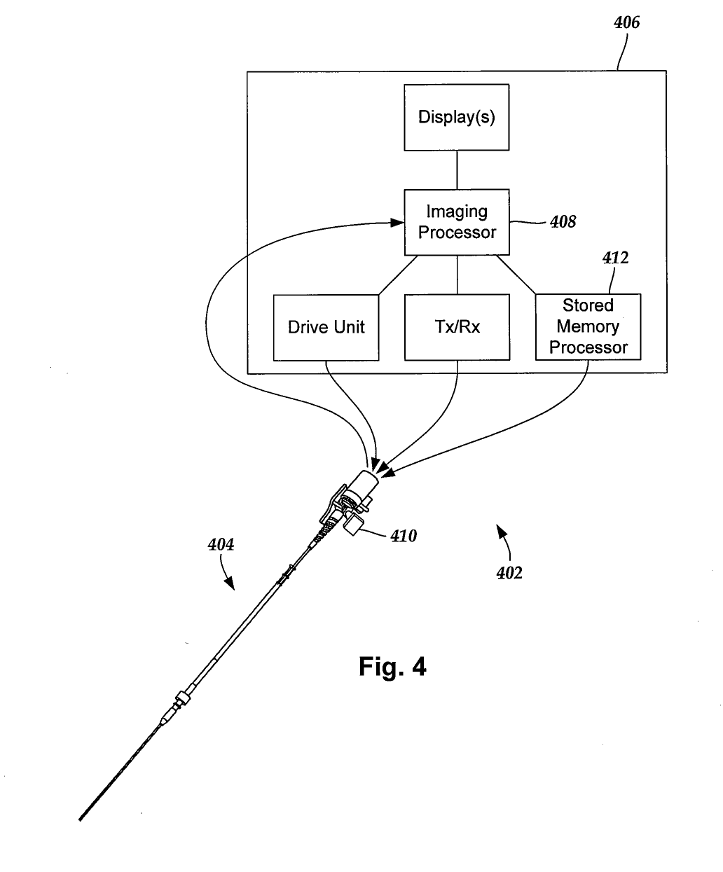

FIG. 4 is a schematic view of another embodiment of a catheter-based imaging system 402. The catheter-based imaging system 402 includes a catheter 404 that is configured and arranged for imaging and that is coupleable to a control module 406 that includes an imaging processor 408. A memory structure 410 is disposed in the catheter 404. The memory structure 410 is coupleable to a data processor 412 configured and arranged to access data from the memory structure 408. In FIG. 4, the data processor 412 is shown coupled directly to the control module 406. As discussed above, however, the data processor 412 can be disposed on a peripheral device that is coupled to the control module 406.

The memory structure 410 is used to store and access catheter management data for the catheter 404. The catheter management data can be stored or accessed at any time. For example, the catheter management data can be stored or accessed during manufacture (e.g., during initial testing during the manufacturing process), prior to an imaging procedure, during an imaging procedure, or during a post-procedure task (e.g., during a failure analysis, or the like). The memory structure 410 can be serially accessed or parallel accessed.

Communication with the memory structure 410 can be performed by any suitable processor including, but not limited to, microprocessors, microcontrollers, or finite state machines. Communication with the memory structure 410 can be via one or more dedicated semiconductors or cores disposed in some form of programmable or custom logic, such as FPGAs or ASICs.

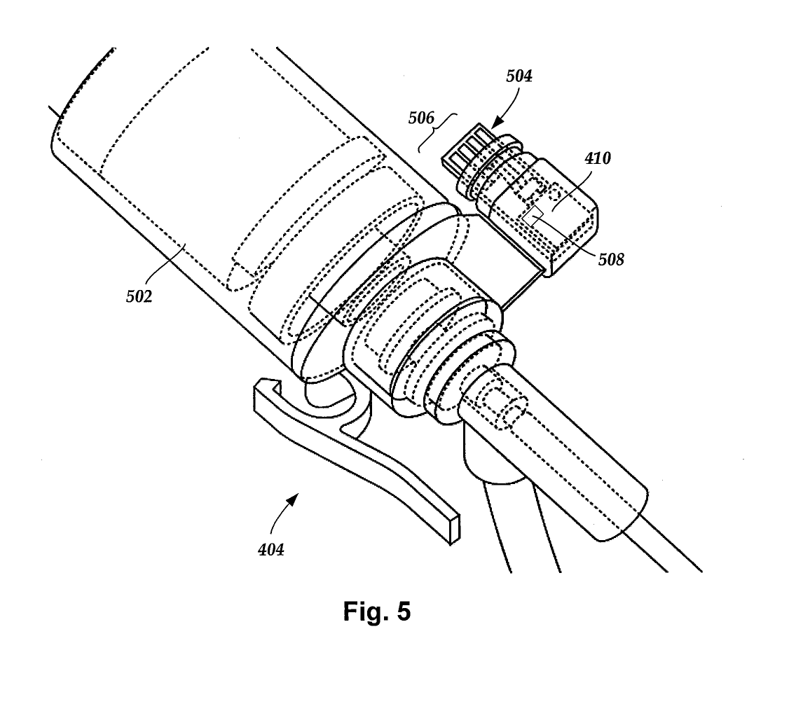

FIG. 5 is a schematic view of one embodiment of the memory structure 410 disposed in a hub 502 of the catheter 404. In FIG. 5, the hub 204 is shown as being semi-transparent around the memory structure 410 for clarity of illustration. Any suitable dedicated non-volatile memory (e.g., EPROM, EEPROM, NVRAM, Flash EPROM, battery-backed SRAM, or the like) can be used in the memory structure 410. Optionally, the memory structure 410 may be writeable after an initial writing of data to the memory structure. In preferred embodiments, the memory structure 410 provides a minimal physical signal count requirement while defining a rich serial communication protocol. A combination of a lower pin count and robust serial communications may enhance the reliability of the interface to read-from and write-to data embedded in the memory structure 410. In at least some embodiments, a 1-Wire® memory (manufactured by Dallas Semiconductor, now a division of Maxim Integrated Products, Sunnyvale, Calif.) is disposed in the hub 502.

The memory structure 410 has an associated interface 504 for coupling to the data processor 412. In one embodiment, the interface 504 has at least four signal traces 506. For example, the interface 504 can include a data/power trace, a ground trace, and at least two traces that provide an electrical loopback function. The two-trace loopback function provides immediate feedback to the catheter-based imaging system (402 in FIG. 4) indicating that the catheter 404 has been coupled to the control module 406. The two-trace loopback function can be used to initiate a serial communication protocol between the memory structure 410 and the data processor 412. It will be understood that any other suitable mechanism or structure can be used to indicate coupling of the catheter 404 to the control module 046. For example, the catheter 404 may include one or more sensors 508 that sense when the catheter 404 is physically coupled to the control module 406. Any suitable sensor(s) can be used including, for example, one or more optical sensors, magnetic sensors (e.g., Hall effect sensors, or the like), capacitive sensors, mechanical sensors (e.g., limit switches, or the like), resistive sensors, or the like.

Catheters are typically sterilized before an imaging procedure. Sterilization processes may include heat, chemicals, or radiation that may corrupt data embedded on the memory structure 410. In at least some embodiments, ethylene oxide can be used to sterilize the catheter 404 and the memory structure 410. In at least some embodiments, the memory structure 410 may be disposed in a shielded enclosure suitable for protecting the memory structure 410 from conditions typically encountered during a sterilization procedure. In at least some embodiments, data is input to the memory structure 410 through a sterile cover after the sterilization procedure. In at least some embodiments, data is input to the memory structure 410 through a sterile cover after the sterilization procedure using wireless communication. In at least some embodiments, the memory structure 410 is removable during the sterilization process.

Once the catheter 404 is coupled to the control module 406 and the memory structure 410 is coupled to the data processor 412, the catheter management data stored in the memory structure 410 can be accessed by the data processor 412 and made available to the catheter-based imaging system 402. In at least some embodiments, the data retrieved from the memory structure 410 is displayed on one or more displays, such as display(s) 112 in FIG. 1, or other coupled displays. In at least some embodiments, cyclic redundancy checks, or other secure means can be used to ensure the correctness of data exchanges between the memory structure 410 and the data processor 412.

The stored catheter management data includes retrievable data that is accessible during a data management communication procedure between the memory structure 410 and the data processor 412. The catheter management data includes data relating to one or more of: 1) verification of the manufacturer of the catheter 404; 2) identification of the catheter 404; 3) functions of the catheter 404 that are available for use during an imaging procedure; 4) manufacturing history of the catheter; 5) prior usage data for the catheter 404; and 6) calibration attributes and parameters for the catheter 404. Additionally, imaging data and system conditions, such as, but not limited to, total imaging time, the length and number of pullbacks, starting and ending catheter core rotational torque measurements, and the like obtained during an imaging procedure can be stored in the memory structure 410 for subsequent retrieval (e.g., during subsequent prior usage data retrieval, during a port-imaging procedure failure analysis, or the like).

In at least some embodiments, the data processor 412 can access pre-defined addresses of the memory structure 410 that contain the name of the manufacturer of the catheter 404. In at least some embodiments, the data management system uses the accessed data to verify that the catheter 404 originated from an approved manufacturer. In at least some embodiments, when the catheter 404 has not originated from an approved manufacturer, the data management communication procedure is discontinued. In at least some embodiments, a registration number may be used as a seed within an authentication algorithm to verify that the catheter 404 originated from an approved manufacturer. In at least some embodiments, an encryption algorithm is defined that writes an encrypted string into the memory during manufacture, and then defines a decryption algorithm resident in the data management system such that the data management system can decipher the encrypted string with the catheter 404 is coupled to the control module 404.

The data processor 412 can access the memory structure 410 to obtain data related to the catheter 404 identification. In some embodiments, further access to the memory structure 410 is available only once the data processor 412 has verified that the catheter 404 is from an approved manufacturer. The catheter identification data may include one or more of the model, type, version, or revision of the catheter 404. In at least some embodiments, the catheter identification data is stored in the memory structure 410 prior to the data management communication procedure. In at least some embodiments, the catheter identification data is stored during manufacturing of the catheter 404.

In at least some embodiments, the data processor 412 includes a database of recognized catheters. In at least some embodiments, the database includes operational attributes and parameters of the recognized catheters. In at least some embodiments, when the catheter 404 is verified, but the catheter 404 is not recognized by the data processor 412 (e.g., the catheter is new, recently updated, or the like), the data processor 412 can access pre-defined addresses of the memory structure 410 that contain the operational attributes and parameters of the catheter 404. In at least some embodiments, the data processor 412 can then update the catheter database with the accessed data. In at least some embodiments, the catheter database can be updated using information from one or more other sources (e.g., the Internet, a CD containing lists of catheters and operational attributes and parameters of the those catheters, or the like).

In at least some embodiments, the catheter management data includes a listing of which catheter functions are available for use during an imaging procedure. For example, two otherwise identical catheters can have one or more different software functions, such as, but not limited to, harmonic imaging, frequency compounding, or the application of specialized post signal processing algorithms used for tissue characterization or lumen detection and the like. The different software functions can be identified via different model numbers. Thus, development and manufacturing costs associated with sometimes providing extraneous functions associated with one-size-fits-all models can be reduced. For example, one or more features of a catheter can be enabled or disabled to meet customer price or usage targets. Additionally, costs may also be reduced for system software development, distribution, service, and maintenance.

In at least some embodiments, the data processor 412 can access the memory structure 410 to obtain data related to the manufacturing history of the catheter 404. The manufacturing history of the catheter 404 can include one or more of, for example, lot codes, manufacturing dates, shelf life end dates, manufacturing locations for the catheter 404, or the like. In at least some embodiments, when the catheter 404 has exceeded its shelf life end date, the data management communication procedure is discontinued. In at least some embodiments, a warning is provided when the catheter 404 has exceeded its shelf life end date. In at least some embodiments, one or more conductor tests may be performed when the catheter 404 has exceeded its shelf life end date to verify whether the catheter is still useable or reliable.

In at least some embodiments, the data processor 412 can access the memory structure 410 to obtain data related to the prior use history of the catheter 404. In at least some embodiments, the memory structure 410 includes a log of the date and start time of a given imaging procedure, the amount of time the catheter 404 was energized, or any other useful or suitable information about the operational history of the catheter 404. In at least some embodiments, the accessed data can be used to disqualify a subsequent use of the catheter 404, or provide a warning to a user, when the time of use exceeds a threshold “safe” duration of time. In at least some embodiments, the use of the catheter 404 may also be disqualified, or a user may be warned, when an attempt is made to reuse a catheter that is not designed for reuse.

In at least some embodiments, the data processor 412 can access the memory structure 410 to obtain data related to proper calibration of the catheter 404. In at least some embodiments, the calibration data is determined and stored in the memory structure 410 during operational tests of the catheter 404 during the manufacturing process. In some embodiments, calibration data may be updated, revised, or stored after the manufacturing process. Calibration data can include data related to improving performance of the catheter 404, such as one or more operational attributes or parameters of the catheter 404, that can be adjusted (via the control module) to improve performance of the catheter-based imaging system 402. For example, the calibration data may be used to modify one or more operational settings of the catheter-based imaging system 402 (e.g., transmit output power, pulse profile, receive path gain sensitivity, or the like or combinations thereof) to improve the image quality of images formed during an imaging procedure.

In at least some embodiments, the calibration data may also include other data, such as transducer sensitivity and bandwidth corner frequencies, that may provide data useful to improve one or more frequency-dependent algorithms used during an imaging procedure (e.g., harmonic imaging, tissue characterization, lumen detection, or the like or combinations thereof). In at least some embodiments, when the operational attributes or parameters of the control module 406 cannot be adjusted to the operational attributes or parameters stored on the memory structure 410, the data management communication procedure is discontinued.

In at least some embodiments, the data processor 412 can record data to the memory structure 410, such as data created during an imaging procedure. The imaging data may include one or more identification numbers for the control module 406 or other peripheral device (e.g., one or more serial numbers, or the like) and one or more error codes (which may be logged during a failure of one or more components during an imaging procedure). The imaging data may also include one or more system conditions (e.g., the time and date of the imaging procedure, the duration of the imaging procedure, catheter rotational motor torque, imaging pullback status, and the like or combinations thereof).

As discussed above, in at least some embodiments the data processor 412 can record data to the memory structure 410 that is related to a catheter failure. For example, in at least some embodiments, the data processor 412 can record one or more system conditions, error codes, usage metrics at failure, system serial numbers, or the like or combinations thereof. For example, the data processor 412 can record one or more of the time, date, or duration of the procedure, catheter rotational motor torque, imaging pullback status, or the like or combinations thereof. In at least some embodiments, at least some of the recorded data can be used to perform a root cause analysis on a failed catheter.

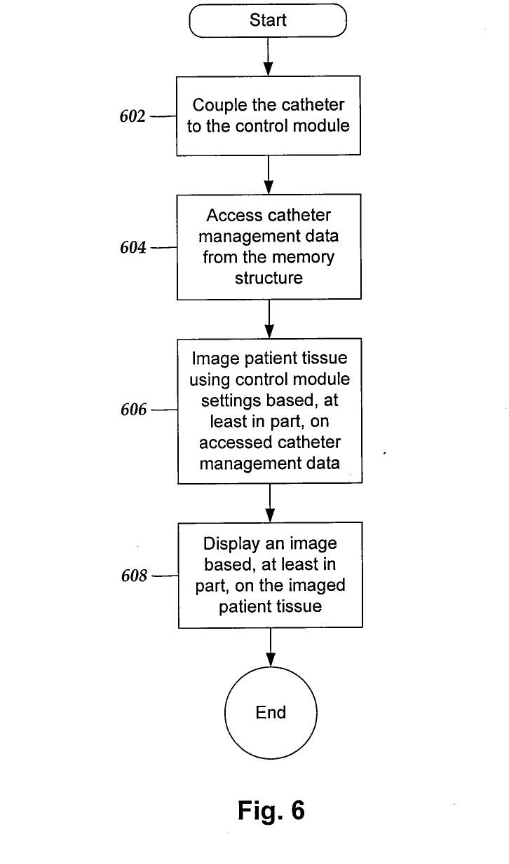

FIG. 6 is a flow diagram showing one exemplary embodiment of a data management communication procedure between the memory structure 410 disposed in the catheter 404 and the data processor 412 coupled to the control module 406. In step 602, the catheter 404 is coupled to the control module 406. In step 604, the catheter management data is accessed from the memory structure 410. In step 606, patient tissue is imaged using control module settings that are selected based, at least in part, on the accessed catheter management data. In step 608 at least one image is displayed based, at least in part, on the imaged patient tissue.

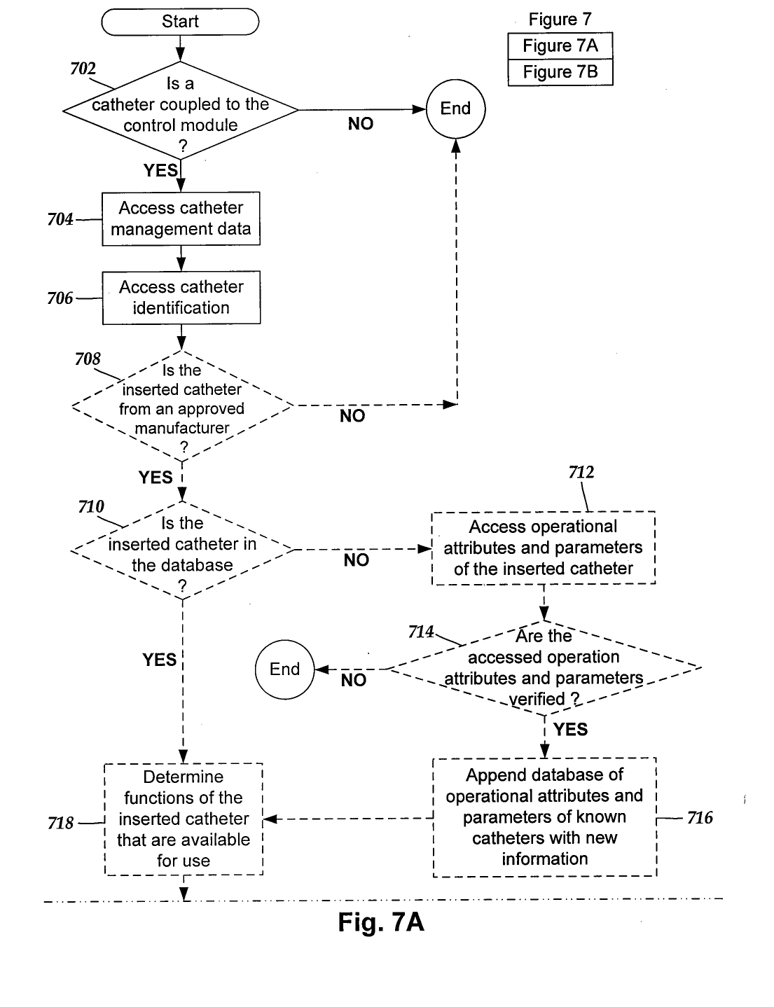

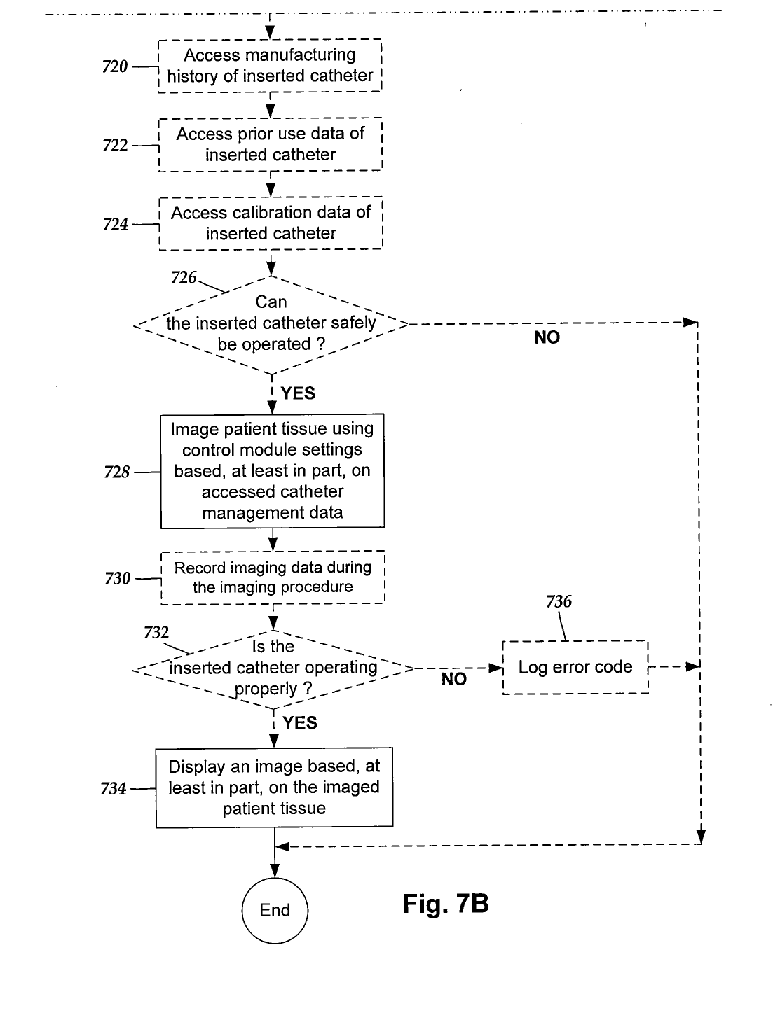

FIG. 7A and FIG. 7B collectively form a flow diagram showing a more detailed exemplary embodiment of a data management communication procedure between the memory structure 410 disposed in the catheter 404 and the data processor 412 coupled to the control module 406. It will be understood that the flow diagram of FIGS. 7A and 7B includes a number of optional steps (shown with dashed lines) that are not necessary to the functioning of the data management communication procedure and that are provided to show an example of how a combination of different catheter management data can be combined together.

When, in step 702, the catheter 404 is not coupled to the control module 406, the data management communication procedure ends. Otherwise, in step 704, catheter management data is accessed (shown in a series of optional steps 708-726). In step 706, the catheter identification data is accessed. In step 708, if the coupled catheter 404 is not manufactured by an approved manufacturer, the data management communication procedure ends, else when, in step 710 the operation attributes and parameters of the catheter 404 are not recognized, control is passed to step 712 where the operation attributes and parameters of the catheter 404 are accessed. In step 714, the accessed data is explicitly verified before the system database is modified, and, in step 716, a database of known catheters and catheter attributes and parameters is updated with the operation attributes and parameters of the catheter 404. Otherwise, in step 718 the available functions of the catheter 404 are determined. In step 720, static system parameters such as serial numbers and date of use are logged into the catheter 404 is accessed. In step 722, the prior use data of the catheter 404 is accessed. In step 724, the calibration data of the catheter 404 is accessed. When, in step 726 it is determined that the catheter-based imaging system 402 cannot be safely operated with the catheter 404, the data management communication procedure ends. Otherwise, in step 728 patient tissue is imaged using control module settings that are selected based, at least in part, on the accessed catheter management data. In step 730, data related to the imaging of patient tissue is written to the memory structure 410. When, in step 732 the catheter 404 finishes imaging the patient tissue without a failure, control is passed to step 734 and at least one image is displayed based, at least in part, on the imaged patient tissue. Otherwise, in step 736 an error code is logged based on the type of failure that occurred.

It will be understood that each block of the flowchart illustrations, and combinations of blocks in the flowchart illustrations, as well any portion of the tissue classifier, imager, control module, systems and methods disclosed herein, can be implemented by computer program instructions. These program instructions may be provided to a processor to produce a machine, such that the instructions, which execute on the processor, create means for implementing the actions specified in the flowchart block or blocks or described for the tissue classifier, imager, control module, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

It will be understood that pullback along one or more of the survey region or the ROI may be performed by pulling the imager from a distal end to a proximal end of the region being imaged. It will also be understood that the intravascular imaging techniques described above can also be used with other types of imaging techniques that use a catheter insertable into patient vasculature. For example, the intravascular imaging techniques can be used with any imaging techniques configured and arranged to assess one or more measurable characteristics of patient tissue (e.g., intravascular magnetic resonance imaging, spectroscopy, temperature mapping, or the like).

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A method of managing catheter data for a catheter-based imaging system, the method comprising:
coupling a catheter to a control module, the catheter comprising a memory structure that comprises catheter management data, the control module comprising a processor;
accessing the catheter management data from the memory structure using the processor;
imaging patient tissue using control module settings that are selected based, at least in part, on the accessed catheter management data; and
displaying at least one image based, at least in part, on the imaged patient tissue.

2. The method of claim 1, wherein the catheter-based imaging system is one of an intravascular ultrasound system or an intracardiac echocardiography system.

3. The method of claim 1, wherein the catheter management data further comprises at least one of catheter verification data, catheter identification data, catheter manufacturing history data, at least one operational attribute or parameter of the catheter, or data related to a suggested lifespan of the catheter.

4. The method of claim 1, further comprising updating a database of recognized and verifiable catheters, as well as operational attributes and parameters of those catheters, by accessing catheter management data of the coupled catheter when the coupled catheter is verifiable, but not recognized, by the processor.

5. The method of claim 1, further comprising writing data to the memory structure that is collected during the imaging of patient tissue.

6. The method of claim 5, wherein writing data to the memory structure that is collected during the imaging of patient tissue comprises recording start and end times of an imaging procedure.

7. The method of claim 5, wherein writing data to the memory structure that is collected during the imaging of patient tissue comprises recording at least one of date, imaging procedure time, catheter rotational motor torque, or imaging pullback status.

8. The method of claim 5, further comprising performing a root cause analysis when the catheter fails, the root cause analysis including at least one or more of logging an error code, accessing system conductions at failure, or accessing data written to the memory structure collected during the imaging of patient tissue.

9. The method of claim 1, wherein imaging patient tissue using control module settings that are selected based, at least in part, on the accessed catheter calibration data comprises adjusting one or more of transmit output power, pulse profile, or receive path gain sensitivity of the catheter-based imaging system.

10. The method of claim 1, wherein imaging patient tissue using control module settings that are selected based, at least in part, on the accessed catheter calibration data comprises recording echo pulse data to permit improvement of frequency dependent algorithms used in at least one of harmonic imaging, tissue characterization, or lumen detection applications.

11. The method of claim 1, further comprising determining catheter functions of the catheter that are available for use during the imaging of patient tissue.

12. The method of claim 1, wherein coupling a catheter to a control module comprises sensing whether the catheter is coupled to the control module using at least one of an optical sensor, magnetic sensor, capacitive sensor, resistive sensor, or a mechanical sensor.

13. A computer-readable medium having processor-executable instructions for reading data from a memory structure disposed on a catheter, the processor-executable instructions when installed onto a device enable the device to perform actions, comprising:
accessing catheter management data from the memory structure;
imaging patient tissue using control module settings that are selected based, at least in part, on the accessed catheter management data; and
displaying at least one image based, at least in part, on the imaged patient tissue.

14. The computer-readable medium of claim 13, wherein the catheter management data further comprises at least one of catheter verification data, catheter identification data, catheter manufacturing history data, at least one operational attribute or parameter of the catheter, or data related to a suggested lifespan of the catheter.

15. The computer-readable medium of claim 13, wherein the actions further comprise updating a database of recognized and verifiable catheters, as well as operational attributes and parameters of those catheters, by accessing catheter management data of the coupled catheter when the coupled catheter is verifiable, but not recognized, by the processor.

16. The computer-readable medium of claim 13, wherein the actions further comprise writing data to the memory structure that is collected during the imaging of patient tissue.

17. A catheter-based imager comprising:
at least one imager and a memory structure disposed in a catheter at least partially insertable into a patient, the at least one imager and the memory structure each coupled to a control module; and
a processor in communication with the control module, the processor for executing processor-readable instructions that enable actions, including:
accessing catheter management data from the memory structure;
imaging patient tissue using control module settings that are selected based, at least in part, on the accessed catheter management data; and
displaying at least one image based, at least in part, on the imaged patient tissue.

18. The computer-readable medium of claim 17, wherein the catheter management data further comprises at least one of catheter verification data, catheter identification data, catheter manufacturing history data, at least one operational attribute or parameter of the catheter, or data related to a suggested lifespan of the catheter.

19. The catheter-based imager of claim 18, wherein the actions further comprise updating a database of recognized and verifiable catheters, as well as operational attributes and parameters of those catheters, by accessing catheter management data of the coupled catheter when the coupled catheter is verifiable, but not recognized, by the processor.

20. The catheter-based imager of claim 17, wherein the actions further comprise writing data to the memory structure that is collected during the imaging of patient tissue.
